(19) United States
(12) Patent Application Publication
(10) Pub. No.: US 2015/0217133 A1
(43) Pub. Date: Aug. 6, 2015

(54) SYSTEM AND METHOD FOR THERAPEUTIC MANAGEMENT OF OCULAR HYPERTENSION

(71) Applicant: Circuit Therapeutics, Inc., Menlo Park, CA (US)

(72) Inventors: David Angeley, Charlottesville, VA (US); Joyce Huang, Redwood City, CA (US); Dan Andersen, Menlo Park, CA (US); Shawnalea Frazier, Menlo Park, CA (US); Griffith Roger Thomas, Burlingame, CA (US)

(73) Assignee: Circuit Therapeutics, Inc., Menlo Park, CA (US)

(21) Appl. No.: 14/592,786

(22) Filed: Jan. 8, 2015

Related U.S. Application Data
(60) Provisional application No. 61/925,104, filed on Jan. 8, 2014.

(51) Int. Cl. A61N 5/06; A61K 38/17; A61K 48/00

(52) U.S. Cl. CPC A61N 5/0622; A61K 48/00; A61K 38/177; A61N 2005/0645

(57) ABSTRACT

One embodiment is directed to a system for treating hypertension within the eye of a patient, comprising a light delivery element optically intercoupled between a light source and a targeted tissue structure of the eye; wherein the targeted tissue structure has been genetically modified to have light sensitive protein; and wherein exposure of the genetically modified targeted tissue structure to radiation delivered through the light delivery element modulates the contractility of the genetically modified targeted tissue structure to reduce an intraocular pressure within the eye.

### RELATED APPLICATION DATA

The present application claims priority to U.S. Provisional Application Ser. No. 61/925,104, filed Jan. 8, 2014. The foregoing application is hereby incorporated by reference into the present application in its entirety.

### FIELD OF THE INVENTION

The present invention relates generally to systems, devices, and processes for facilitating various levels of control over cells and tissues in vivo, and more particularly to systems and methods for physiologic intervention wherein trabecular meshwork contractility is modulated to treat raised intraocular pressure. Among the approaches that are envisioned by the present invention to alter trabecular meshwork contractility include pharmacological agents to alter endogenous pathways influencing trabecular cell contractility and biological treatments such as gene therapy to modulate the contractility of the trabecular meshwork for the treatment of intraocular pressure. Gene therapy may introduce genes which can allow unregulated, sustained alteration in trabecular contractility or may block endogenous genes using siRNA or other genetic methods to block endogenous gene expression to alter trabecular meshwork contractility to reduce raised intraocular pressure. Gene therapy may also introduce genes which permit regulated alteration in trabecular cell contractility in response to an exogenous agent or stimulus, including pharmacological or biological agents, or stimuli such as light, electricity, pressure, irradiation or ultrasound. In one embodiment where opsins are the gene delivered via the gene therapy light may be utilized as an input to tissues which have been modified to become light sensitive. In another embodiment of the invention, where pharmacologically-activated exogenous receptors are utilized to modulate the contractility of the trabecular meshwork designer agonists may be used to activate the genetically modified cells.

### BACKGROUND

Glaucoma is the second-leading cause of blindness in the world, with cataracts being the first. Glaucoma is also the leading cause of blindness in African Americans. As of 2010, there were 44.7 million people in the world with open angle glaucoma, 2.8 million of them in the United States. By 2020, the prevalence is projected to increase to 58.6 million worldwide and 3.4 million the United States. Glaucoma can be roughly divided into two main categories, “open-angle” and “closed-angle” (or “angle closure”) glaucoma. Referring to FIG. 1, an anatomical diagram depicting features of the human eye is shown. In reference to glaucoma, the “angle” refers to the space between the iris and cornea, through which fluid (aqueous humor (AH)) must flow to drain from the eye via the trabecular meshwork (TM). Closed-angle glaucoma can appear suddenly and is often painful; visual loss can progress quickly, but the discomfort often leads patients to seek medical attention before permanent damage occurs. Open-angle, chronic glaucoma tends to progress at a slower rate and patients may not notice they have lost vision until the disease has progressed significantly. The exact etiology of open-angle glaucoma remains unknown. However, the major risk factor for most glaucoma patients, and the focus of treatment, is increased intraocular pressure (IOP), i.e. ocular hypertension (OHT). A progressive loss of the visual field due to cell loss in the retinal nerve fiber layer is a direct result of OHT. Vision loss can negatively affect a patient's quality of life and mobility, such as the ability to drive, which has a severe negative macroeconomic impact. The present invention relates predominantly to the treatment of OHT in open-angle glaucoma.

IOP is mainly maintained by the aqueous humor, which is produced by the ciliary body of the eye. When the ciliary bodies produce the aqueous humor, it first flows into the posterior chamber (bounded by the lens and the iris). It then flows through the pupil of the iris into the anterior chamber (bounded by the iris and the cornea). From here, it flows through the TM to enter the normal body circulation via Schlemm's canal (SC), also known as the scleral venous sinus, which is a circular channel in the eye that collects aqueous humor from the anterior chamber and delivers it into the bloodstream via the anterior ciliary veins. The canal is essentially an endothelium-lined structure, resembling that of a lymphatic vessel. In the human eye, the SC transfers an average of approximately 3 μl of aqueous humor per minute. Thus, the IOP is maintained by a delicate balance between synthesis and drainage of AH. Therefore, a decrease in outflow through the trabecular meshwork or uveoscleral pathways increases IOP resulting in OHT. The primary outflow pathway is via the TM which also makes the greatest contribution to outflow resistance of the AH, and is the therapeutic focus of the present invention.

The modern goals of glaucoma management are to avoid glaucomatous damage and nerve damage, and preserve visual field and total quality of life for patients, with minimal side effects. Screening for glaucoma is usually performed as part of a standard eye examination, which should include measurements of the IOP via tonometry. The retinal nerve fiber layer can be assessed with imaging techniques such as optical coherence tomography, scanning laser polarimetry, and/or scanning laser ophthalmoscopy.

Generally IOP may be lowered with medication, usually in the form of eye drops. Several different classes of medications have been used, with several different medications in each class. Often the therapeutic effect of these medicines may be limited by local and systemic side effects. If side effects occur, the patient must be willing to either tolerate them, or to communicate with the treating physician to improve the drug regimen. Poor compliance with medications and follow-up visits has been cited as a major reason for vision loss in glaucoma patients.

Both laser and conventional surgeries have been performed to treat OHT, especially for those with congenital glaucoma. Although they have high success rates, these operations generally represent a temporary solution, with re-treatments required periodically, such as biennially. In most cases, medications are still necessary to control and maintain post-op IOP. However, surgery may lessen the amount of medication needed.

Thus, there remains a need for robust and reliable therapies for the treatment of OHT by relaxing the cells of the TM to lower IOP by reducing the hydraulic impedance of the TM and/or the hydraulic impedance of entering and being conducted along Schlemm's Canal to the outflow of AH. It is known that the state of contraction of the TM influences the shape of and therefore fluid flow within Schlemm's Canal.

As is described in Stumpff and Wiederholt in Ophthalmologica 2000; 214:33-53, which is incorporated by reference herein in its entirety, in the traditional concept, trabecular meshwork is an inert tissue, with no regulatory properties of its own. In this concept, regulation of outflow resistance is determined by the ciliary muscle. However, work done during the last decade has established that, in addition to being passively distended by the ciliary muscle, the trabecular meshwork has contractile properties of its own. Specifically, the contraction and relaxation of this structure may influence ocular outflow in the sense that relaxation reduces intraocular pressure. Ample evidence supports the theory that trabecular meshwork possesses smooth-muscle-like properties. In addition, trabecular meshwork cells express a large number of transporters, channels and receptors, many of which are known to regulate smooth-muscle contractility. It has been shown that trabecular meshwork can be induced to contract and relax in response to pharmacological agents. Agents that contract trabecular meshwork reportedly reduced outflow in some primate experiment. On the cellular level, this is coupled with depolarization of the plasma membrane and a rise in intracellular calcium. Relaxation of trabecular meshwork, on the other hand, appears to be coupled to a stimulation of the maxi-K channel, inducing hyperpolarization of the plasma membrane and the inactivation of L-type calcium channels.

Described herein are configurations for bringing about TM cell relaxation. These mechanisms are configured to decrease the contractility of the TM cells by either reducing the availability of intracellular calcium or attenuating the ability of the cell to utilize intracellular calcium to activate contractile elements within the cell.

### The Opsins

Certain light-activated ion pumps may be used to induce hyperpolarizing the TM cells thus attenuating the opening of L-type calcium channels. This will reduce the levels of intracellular calcium which in turn will diminish the contractility of the TM cells. In the context of optogenetic application, the expression-enhanced version of a halorhodopsin called NpHR (derived from the halobacterium Natronomonas pharaonis), acts as an electrogenic chloride pump acts to increase the separation of charge across the plasma membrane of the targeted cell upon activation by yellow light. NpHR may be characterized as a true pump that requires constant light to move through its photocycle. Since 2007, a number of modifications to NpHR have improved its function. Codon-optimization of the DNA sequence followed by enhancement of its subcellular trafficking (eNpHR2.0 and eNpHR3.0) resulted in improved membrane targeting and higher currents more suitable for use in mammalian tissue. A new class of channel, recently described by Karl Deisseroth et al, such as in Science. April 2014. 344(6182):420-4, which is incorporated by reference in its entirety, is based on ChR but is modified to permit cations to pass through the channel, rather than anions. In response to blue light, this new “inhibitory” channel (which may be termed “iChR” or “SwiChR”) will open and permit large amounts of Cl— ions to pass, thereby hyperpolarizing the neuron more effectively and thus inhibiting the cell with greater efficiency and sensitivity.

Proton pumps archaerhodopsin-3 (“Arch”) and “eARCH”, and ArchT, Leptosphaeria maculans fungal opsins (“Mac”), enhanced bacteriorhodopsin (“eBR”), and Guillardia theta rhodopsin-3 (“GtR3”) have also been developed as optogenetic tools and claimed herein as optogenetic proteins that when activated by light may be used to hyperpolarize the TM cells by pumping hydrogen ions out of the cell. Membrane hyperpolarization produced via this mechanism may lead to reduced contractility in a manner similar to the mechanism described above.

### Pharmacogenetic-Based Therapeutics.

Certain modified pharmacologically-activated receptors also may be used to bring about hyperpolarization of the TM cells. Such pharmacological actuators have been engineered for selective activation by a synthetic/biologically inert compound with simultaneous abolishment of any sensitivity to endogenous agonists. For example activation the modified Gi protein-coupled human muscarinic M4 receptor (hM4Di) by its orthogonal agonist ligand (clozapine-N-oxide (CNO)) leads to a hyperpolarization of the cell via activation of a GIRK potassium channel. As described above for theopsins such hyperpolarization may lead to a decreased opening of L-type calcium channels thereby reducing intracellular calcium levels and attenuating the contractile response/tone.

In respect to other G-protein-coupled pathways, smooth muscle cells are also often relaxed by receptor-induced activation of adenylyl cyclase. In some smooth muscle, as in the lung for example, beta-2-adrenoceptor agonists activate adenylyl cyclase which catalyzes the production of cyclic adenosine monophosphate (cAMP). Elevated cAMP levels leads to activation of protein kinase A, which activates potassium channels to hyperpolarize the membrane while concomitantly phosphorylating other proteins (e.g. myosin light chain kinase and phospholamban) to prevent contraction and enhance reuptake of Ca²⁺ into the sarcoplasmic reticulum thereby lowering intracellular Ca²⁺ levels. A similar effect may be brought about in the TM cells by expression of the modified Gs-coupled rat muscarinic M3 receptor (rM3Ds) and its subsequent selective activation using its orthogonal agonist ligand CNO.

Presented herein are approaches that utilize the tissue-specific delivery; cell selective expression and subsequent receptor-selective activation of naturally-occurring genes that encode exogenous receptor genetic material, such as light-sensitive transmembrane proteins (or “opsins”), or pharmacologically-activated exogenous receptors (or “pharmacogenetics”), such as “designer receptors exclusively activated by designer drugs” (DREADDs), the modified Caenorhabditis elegans glutamate-gated chloride channel (GluCl), or “pharmacologically selective actuator modules” (PSAMs), by way of non-limiting example, to bring about relaxation of the contractile elements within the TM cells; increasing the permeability of the TM tissue structures resulting in a reduction in hydraulic impedance and thereby reducing high IOP to control OHT.

### SUMMARY

One embodiment is directed to a system for treating hypertension within the eye of a patient, comprising a light delivery element optically intercoupled between a light source and a targeted tissue structure of the eye; wherein the targeted tissue structure has been genetically modified to have light sensitive protein; and wherein exposure of the genetically modified targeted tissue structure to radiation delivered through the light delivery element modulates the contractility of the genetically modified targeted tissue structure to reduce an intraocular pressure within the eye. The light delivery element may comprise a contact lens configured to directly interface with a corneal surface of the eye. The contact lens may be configured to direct light incoming toward the eye to a structure selected from the group consisting of: a trabecular meshwork of the eye; and a schlemm's canal of the eye. The contact lens may be configured to direct light incoming toward the eye through an intermediate tissue structure selected from the group consisting of: a cornea of the eye; a limbus of the eye; and a sclera of the eye. The contact lens further may be configured to comprise an optical element or segment configured to reflect incoming light. The contact lens further may be configured to comprise an optical element or segment configured to focus incoming light. The contact lens further may be configured to comprise an optical element or segment configured to both reflect and focus incoming light. The contact lens further may be configured to comprise a spectral filtering element. The light delivery element may comprise an intraocular lens configured to redirect at least a portion of radiation incoming toward the eye to the targeted tissue structure of the eye. The intraocular lens may comprise an implantable prosthesis. The intraocular lens may be configured to direct light incoming toward the eye to a structure selected from the group consisting of: a trabecular meshwork of the eye; and a schlemm's canal of the eye. The intraocular lens may be configured to direct light incoming toward the eye through an intermediate tissue structure selected from the group consisting of: a cornea of the eye; a limbus of the eye; and a sclera of the eye. The intraocular lens further may be configured to comprise an optical element or segment configured to reflect incoming light. The intraocular lens further may be configured to comprise an optical element or segment configured to focus incoming light. The intraocular lens further may be configured to comprise an optical element or segment configured to both reflect and focus incoming light. The light source may be coupled to a mounting structure configured to be removably coupled to the patient. The mounting structure may comprise an eyeglasses frame. The light source may comprise ambient light from an environment around the patient. The light sensitive protein may be an opsin protein. The opsin protein may be an inhibitory opsin protein. The inhibitory opsin protein may be selected from the group consisting of: NpHR, eNpHR 1.0, eNpHR 2.0, eNpHR 3.0, Mac, Mac 3.0, Arch, ArchT, and iChR. The opsin protein may be a stimulatory opsin protein. The stimulatory opsin protein may be selected from the group consisting of: ChR2, C1V1-T, C1V1-TT, CatCh, VChR1-SFO, and ChR2-SFO. The light source may be configured to produce pulses of light to be delivered to the targeted tissue structure of the eye.

### BRIEF DESCRIPTION OF THE DRAWINGS

FIG. 1 illustrates certain aspects of the anatomy of the human eye.

FIG. 2 illustrates certain aspects of a treatment paradigm in accordance with the present invention.

FIG. 3 illustrates a plot of irradiance versus wavelength.

FIG. 4 illustrates a comparative table illustrating aspects of various light-sensitive opsin proteins.

FIG. 5 illustrates an embodiment of a contact lens configuration.

FIG. 6 illustrates an embodiment of a contact lens configuration.

FIG. 7 illustrates an embodiment of a contact lens configuration.

FIGS. 8A and 8B illustrate aspects of an embodiment of a contact lens configuration.

FIG. 9 illustrates an embodiment featuring a wavelength-specific blocking/filtering functionality.

FIGS. 10A and 10B illustrate embodiments of contact lens configurations.

FIG. 11 illustrates elements of a system configured to practice an embodiment of the present invention.

FIGS. 12A and 12B illustrate embodiments of the present invention.

FIG. 13 illustrates a schematic representation of an embodiment of the present invention.

FIG. 14 illustrates an embodiment of the present invention.

FIG. 15 illustrates a block diagram of a system configured to practice the practice the present invention.

### DETAILED DESCRIPTION

As described briefly above, central to the present invention is the use of gene therapy to deliver exogenous genetic material to controllably modulate the contractility of TM cells to reduce the resistance to AH flow through the TM tissue structures to treat OHT. In one group of embodiments, the exogenous receptor material may be selected to encode light-sensitive proteins; in another group of embodiments, the exogenous receptor material may be selected to encode pharmacologically-activated exogenous receptors, such as DREADDs, GluCl, or PSAM-GlyR, by way of non-limiting example; in a further group of embodiments, both light-sensitive protein and pharmacogenetic techniques may be combined.

Cellular specificity may be obtained with viruses by virtue of delivery route, virus type and serotype selection, promoter selection as discussed in further detail below, and by restriction of opsin activation (i.e., via targeted illumination) of particular cells or selective delivery of orthogonal agonist ligands to activate modified receptors, also as described in further detail below.

### Route of Delivery:

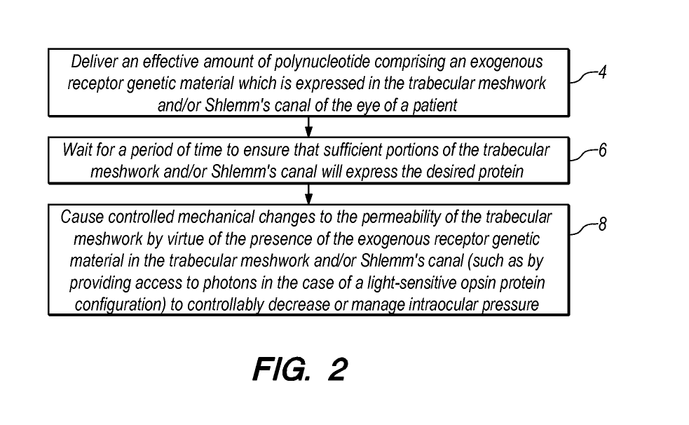

Referring to FIG. 2, in each configuration, generally the genetic material first may be delivered, followed by confirmation of expression in the desired cells. Delivery of the selected exogenous material to the eye of the patient may follow one or more paradigms, such as those described below, which may take advantage of the unique anatomical positioning/access of the human eye relative to other systems and/or structures.

As is described in Buie, et al. 2010 IOVS, 51; 1:236-48, which is incorporated by reference herein in its entirety, the location, morphology, and physiology of the cells of the outflow pathway of the eye lend themselves to efficient gene delivery. Because of the natural flow of aqueous humor, genes delivered into the anterior chamber may preferentially reach the trabecular meshwork. Once the vectors reach the trabecular meshwork, the physiological flow pattern of the fluid between and around the trabecular meshwork cell layers may provide the transfer molecules with a longer contact time and may facilitate their entry into the cells.

Referring back to FIG. 2, delivery of the polynucleotide comprising the exogenous receptor genetic material to be expressed in cells of the targeted anatomy may involve injection with a syringe or other device, in one or more configurations, including but not limited to internal topical injection or application (i.e., injection upon a surface of a tissue structure associated with a targeted portion of anatomy, or upon the anatomy itself, generally after surgical access, such as via endoscopic techniques). Each of these injection configurations is explored in further detail below.

Intracameral administration or application to a tissue structure surface may be utilized to deliver genetic material for optogenetic or pharmacogenetic therapy. Recombinant vectors are capable of diffusing through tissues and infecting cells following such topical application or exposure. The efficacy of topical application of viral vectors has been increased using vector solutions suspended in gels. In one embodiment, a vector may be suspended in a gel and applied to the surface of tissues, or placed in the same anatomical space as the target tissue. Internal topical application may be achieved using laparoscopic techniques, wherein one or more small incisions may be made through the outer layer(s) of the eye and other pertinent tissue structures to allow insertion of the surgical apparatus (camera, needle, tools, etc.). A needle may be inserted intracamerally (as visualized through the camera or other imaging devices, such as a slit lamp biomicroscope, or operating microscope). In all cases, the vector may be mixed with a gel (e.g. the products sold under the tradenames “Healon” by Abbott, or “Viscoat” by Alcon) and then sprayed onto, painted onto, or injected out upon the surface of the pertinent tissue. A dose of approximately 0.1 mL saline containing 1×10¹¹ vg of AAV may be used to cover each 1 cm² area. These ranges are illustrative, and doses are tested for each virus-promoter-opsin/receptor construct pairing them with the targeted TM cells.

In one particular example of topical application, ocular hypertension may be addressed by topical application of vector solution or gel within the anterior chamber of the eye using a needle under microscopic visualization to achieve transfer of optogenetic material to the pertinent cells. The vector may be applied directly and topically at either as a bolus into the aqueous humor of the anterior chamber or at multiple sites nearby the TM to cover as much of the available TM surface as possible, the goal being to infect the cells of the TM. Alternately, a plug of virus-laden gel may be placed in the anterior chamber and allowed to elute virus over the course of several hours. The plug should be placed such that it does not substantially occlude the TM, however. In a further alternate embodiment, a virus-eluting trabecular plug may be inserted for similar effect.

An ophthalmic balanced salt solution, such as BSS, by Alcon, may be used to prepare the vector injection.

Access to the anterior chamber (AC) may be made after instillation of a topical anesthetic, such as proparacaine (sold as Alcaine, by Alcon) and a lid speculum may be inserted, such as the Seibel 3-D Lid Speculum, by Storz, to allow for a needle injection to be made into the AC. Alternately, in lieu of a needle injection, a paracentesis may be performed at the superior temporal limbus by using a sharp stab blade, such as the MIP Diamond Knife, by ASICO. An amount of aqueous humor may be discharged, and the vector injection may be performed using, for example, a 25 to 30-gauge anterior chamber cannula, such as a blunt-tipped Knolle Anterior Chamber Irrigating Cannula, by Storz, that is introduced into the AC via the paracentesis. Alternately, displaced aqueous humor may be vented intra-operatively via a paracentesis.

### Viral Vectors:

Viral expression systems have the dual advantages of fast and versatile implementation combined with high infective/copy number for robust expression levels in targeted anatomy. Viral expression techniques, such as those comprising delivery of DNA encoding a desired opsin/promoter/catalyst sequence packaged within a recombinant viral vector, have been utilized with success in mammals to effectively transfect a targeted anatomy. They deliver genetic material to the nuclei of targeted cells, thereby inducing such cells to produce light-sensitive proteins. The proteins are then transported to the cell membranes where they are made functionally available to illumination components of the interventional system. In the case of a light-sensitive protein configuration, typically a viral vector will package what may be referred to as an “opsin expression cassette”, which may contain the opsin (e.g., Arch, NpHR, etc.) and a promoter that may be selected to drive expression of the particular opsin. In the case of Adeno-associated virus (or AAV), the gene of interest (in this example an opsin) may be in a full-length genomic configuration with only one opsin expression cassette in a standard AAV backbone of approximately 5000 bases. An alternative embodiment utilizes a self-complementary AAV structure with two copies of opsin expression cassette complimentary in sequence with one another and connected by hairpin loops are encapsulated within the viral envelope, in order to avoid the need for full-length vectors to synthesize a second copy of complementary DNA.

In one embodiment, a gene product may be targeted by methods described in Yizhar et al. 2011, Cell 71:9-34, which is incorporated by reference herein in its entirety. In addition, different serotypes of the virus (conferred by the viral capsid or coat proteins) will show different tissue tropism. Lenti- and adeno-associated (“AAV”) viral vectors have been utilized successfully to introduce opsins into the mouse, rat and primate brain and the eye (for example, as described by Borrás, T., C. Brandt, et al. (2002). “Gene therapy for glaucoma: treating a multifaceted, chronic disease.” Invest Ophthalmol Vis Sci. 43(8): 2513-2518, which is incorporated by reference herein in its entirety). Additionally, these have been well tolerated and highly expressed over relatively long periods of time with no reported adverse effects, providing the opportunity for long-term treatment paradigms.

Viruses have been utilized to target many tissue structures and systems, including but not limited to ciliary epithelium, ciliary muscle retinal ganglion cells as well as trabecular meshwork cells. To date, six delivery systems have been tested for ability to deliver genes to the relevant tissues or cells. These include adenoviruses (Ads), adenoassociated viruses (AAVs), herpes simplex viruses (HSVs), lentiviruses (LVs; feline immunodeficiency virus [FIV] and human immunodeficiency virus [HIV]), liposomes (LPs), and naked DNA. Of these, AAV is a preferred vector due to its safety profile. However, published literature reports that standard, full-length AAV does not successfully infect TM cells and that these can only be infected using self-complementary AAV. Our data show that AAV 1, AAV5 and AAV6 are capable of successfully infecting TM cells, although full-length AAVs of other serotypes may be utilized as well. These full-length AAVs are preferable to self-complementary AAVs since they allow twice of the length of DNA to be inserted into the AAV backbone as only one copy of the gene is needed. This would be particularly useful for the use of cell-type specific promoters, which often require a certain length of promoter sequence which cannot fit within the constraints of self-complementary AAV. Since prior teaching reported that full-length AAV cannot be used for gene transfer to trabecular meshwork cells, our results represent a novel inventive step enabling the use of these superior, full-length AAV vectors for gene transfer to trabecular cells. Nonetheless, for those genes of sufficiently small size, such as opsin genes, and those instances when cell-type specific promoters are not necessary, self-complementary AAVs may be used to achieve the desired therapeutic outcome and are envisioned as embodiments of the present invention.

### Promoters:

The promoter may confer specificity to a targeted tissue, such as in the case of the human synapsin promoter (“hSyn”) or the human Thy1 promoter (“hThy1”) which allow protein expression of the gene under its control only in specific cell types (i.e. neurons). Another example is the calcium/calmodulin-dependent kinase II promoter (“CAMKII”), which allows protein expression of the gene under its control only in excitatory cells, a subset of the cell population. Alternatively, a ubiquitous promoter may be utilized, such as the human cytomegalovirus (“CMV”) promoter, or the chicken beta-actin (“CBA”) promoter, each of which is not particularly specific, and each of which has been utilized safely in gene therapy trials. Alternatively, a combination of chicken beta-actin promoter and cytomegalovirus immediate-early enhancer, known as CAG promoter, may be utilized. Alternatively, a human elongation factor-1 alpha EF1α promoter may be utilized, including for example those from isoforms EF1α₁ and EF1α₂. EF1α₁ promoter may confer expression in brain, placenta, lung, liver, kidney, and pancreas. EF1α₂ promoter may confer expression in terminally differentiated cells of the brain, heart, and skeletal muscle as well, as we have found, TM cells.

Targeted gene expression via AAV-mediated gene transfer into the TM cells of the outflow pathway has previously been demonstrated using promoter fragments from the matrix Gla protein (MGP) gene (Gonzalez et al., (2004) Expression analysis of the matrix Gla protein and VE-cadherin gene promoters in the outflow pathway. Invest Ophthalmol Vis Sci. 45:1389-1395; incorporated by reference herein in its entirety). Selective targeting has also been achieved using the 5′ promoter region of the chitinase 3-like 1 (“Ch3L1”) gene, with expression specifically directed to the outermost anterior and posterior regions of the TM (Liton et al., (2005) Specific targeting of gene expression to a subset of human trabecular meshwork cells using the chitinase 3-like 1 promoter. Invest Ophthalmol Vis Sci. 46:183-190; incorporated by reference herein in its entirety). Further, numerous gene profiling studies of the trabecular meshwork have been published, providing additional alternative configurations for trabecular meshwork cell-selective promoters (Gonzalez et al., (2000) Characterization of gene expression in human trabecular meshwork using single-pass sequencing of 1060 clones. Invest Ophthalmol Vis Sci. 41:3678-3693; Wirtz et al., (2002) Expression profile and genome location of cDNA clones from an infant human trabecular meshwork cell library. Invest Ophthalmol Vis Sci. 43:3698-3704; Tomarev et al., (2003) Gene expression profile of the human trabecular meshwork: NEIBank sequence tag analysis. Invest Ophthalmol Vis Sci. 44:2588-2596; Liton et al., (2005) Specific targeting of gene expression to a subset of human trabecular meshwork cells using the chitinase 3-like 1 promoter. Invest Ophthalmol Vis Sci. 46:183-190; Liton et al., (2006) Genome-wide expression profile of human trabecular meshwork cultured cells, non glaucomatous and primary open angle glaucoma tissue. Mol Vis. 12:774-790; Fan et al., (2008) Gene expression profiles of human trabecular meshwork cells induced by triamcinolone and dexamethasone. Invest Ophthalmol Vis Sci. 49:1886-1897; Fuchshofer et al., (2009) Gene expression profiling of TGFuman trabecular meshwork cells induced by triamcinolonetification of Smad7 as a critical inhibitor of TGF-β2 signaling. Exp Eye Res. 88:1020-1032; Paylakhi et al., (2012) Non-housekeeping genes expressed in human trabecular meshwork cell cultures. Mol Vis. 18:241-254; and Liu et al., (2013) Gene expression profile in human trabecular meshwork from patients with primary open-angle glaucoma. Invest Ophthalmol Vis Sci. 54:6382-6389; each of which is incorporated by reference herein in its entirety).

### Gene Products Responsive to Pharmacological Activation:

In embodiments wherein pharmacogenetic techniques are to be utilized to manage IOP by modulating the resistance to AH flow (i.e., in the TM), targeted cells may be genetically engineered or modified to respond to specific chemical ligands to provide such pharmacological control. For example, cells within the trabecular meshwork may be modified with a nucleic acid comprising a nucleotide sequence encoding an orthogonal receptor, such as those described by Shapiro et al (ACS Chemical Neuroscience, 2012, 3, pgs 619-629, which is incorporated by reference herein in its entirety), including by not limited to those referred to as DREADDs, GluCl, or PSAM-GlyR, which may be selected specifically for their orthogonality (i.e., they respond to specific chemical ligands that have no interfering pharmacological effect).

Following infection of the TM cells a period of time may be necessary for expression of the encoded proteins. Once expressed the proteins must be activated to bring about relaxation of the TM. In one embodiment the pharmacogenetic receptors (e.g. DREADDs) may be activated by their designer agonist clozapine-N-oxide (CNO). CNO can either be given systemically (preferably orally (PO) as a tablet) or delivered locally to the eye, by way of non-limiting examples. Local eye delivery may be accomplished by the use of drops that are administered regularly to the surface of the cornea as is the case with current “eye drop” administrations. The timing of the administration and the dose delivered may be calculated to ensure that a constant pharmacologically active concentration of CNO is present in the anterior chamber of the eye. This concentration may be expected to be in the order of 1 to 1000 μM. Various approved ophthalmic formulations may be employed to optimize the delivery of the ligand as well as experimental delivery systems such as those described by Gooch et al (2012) Ocular drug delivery for Glaucoma management, Pharmaceutics 4, 197-211, which is incorporated by reference herein in its entirety. These may include but not be restricted to conjunctival, subconjunctival, and intravitreal inserts, punctal plugs, and drug depots.

What is described as examples of delivery systems for CNO above (as the orthogonal agonist of the DREADD receptors) also applies to orthogonal agonists of the modified Caenorhabditis elegans glutamate-gated chloride channel (GluCl)—Ivermectin, or “pharmacologically selective actuator modules” (PSAMs)—“pharmacologically selective effector molecule” (PSEM).

### Gene Products Responsive to Light Activation:

In embodiments wherein opsins are to be utilized to create changes in the resistance to AH flow by modulating the contractility of the cells of the TM, as described herein, selected light-sensitive transmembrane proteins may be covalently bonded to chromophore retinal, which upon absorption of light, isomerizes to activate the protein. Notably, retinal compounds are found in most vertebrate cells in sufficient quantities, thus eliminating the need to administer exogenous molecules for this purpose. The first genetically encoded system for optical control in mammalian cells using light-sensitive signaling proteins was established in Drosophila melanogaster, a fruit fly species, and cells expressing such proteins were shown to respond to light exposure with waves of depolarization and spiking. More recently it has been discovered that opsins from microorganisms which combine the light-sensitive domain with an ion pump or ion channel in the same protein may also modulate cell signaling to facilitate faster control in a single, easily-expressed, protein. Other opsin configurations have been found to directly inhibit signal transmission without hyperstimulation or overdriving. For example, light stimulation of halorhodopsin (“NpHR”), a chloride ion pump described above, hyperpolarizes cells in response to yellow-wavelength (˜589 nm) light irradiation. Other more recent variants (such as those termed “eNpHR2.0” and “eNpHR3.0”) exhibit improved membrane targeting and photocurrents in mammalian cells. Light driven proton pumps such as archaerhodopsin-3 (“Arch”) and “eARCH”, and ArchT, Leptosphaeria maculans fungal opsins (“Mac”), enhanced bacteriorhodopsin (“eBR”), and Guillardia theta rhodopsin-3 (“GtR3”) may also be utilized to hyperpolarize cells and block signaling. Direct hyperpolarization is a specific and physiological intervention that mimics normal cellular inhibition. Suitable inhibitory and stimulatory opsins are described, for example, in PCT/US2013/000262, which is incorporated by reference herein in its entirety. As described in the aforementioned incorporated reference, the term light-sensitive protein, as used herein, refers to all the aforementioned types of ion channels and ion transporters/pumps in the context of modulating a membrane potential. In one embodiment, as described above, in response to blue light, an “inhibitory” channel (such as those referred to as “iChR” or “SwiChR”) may be utilized to open and permit large amounts of Cl— ions to pass, thereby hyperpolarizing the neuron more effectively and thus inhibiting the cell with efficiency and sensitivity.

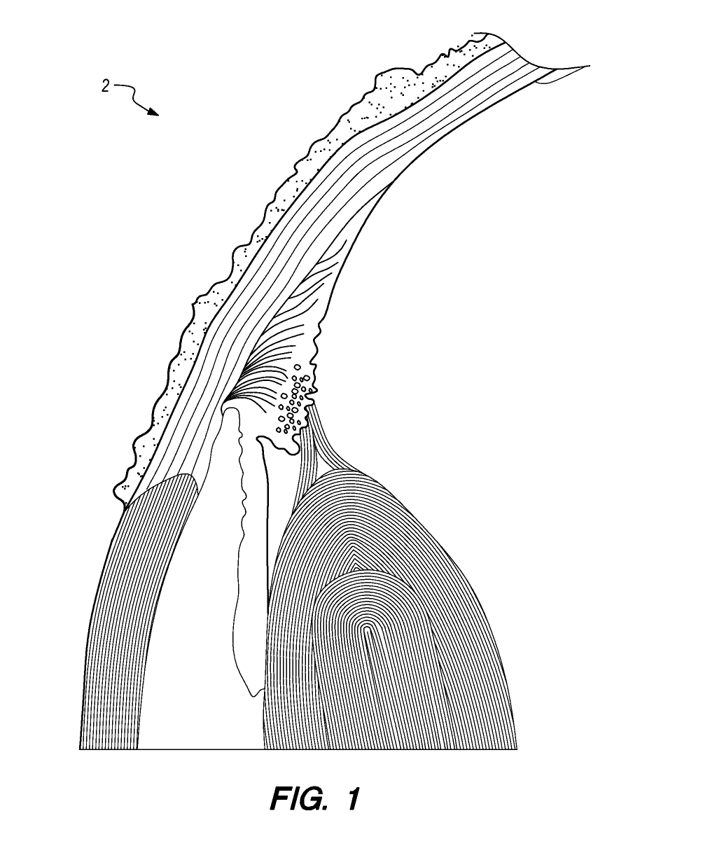

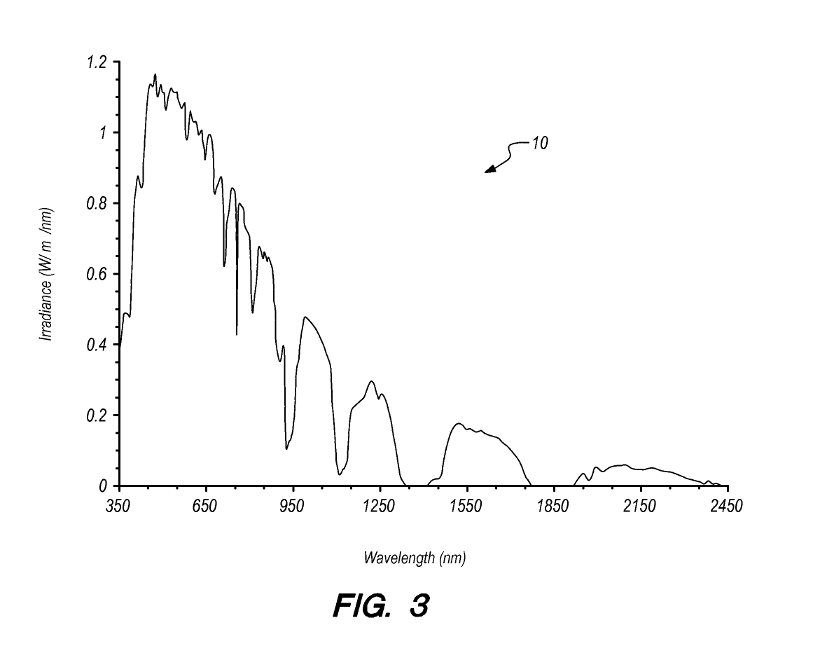

The activation spectrum of the opsin (light-sensitive configuration) NpHR, by way of non-limiting example, is about 100 nm wide and centered at about 580 nm. The nominal solar spectral irradiance at sea level across this spectral region is about 1.1 W/m²/nm, as shown in FIG. 3. This yields an unaided, response-weighted irradiance at the ocular surface of about 0.1 mW/mm² within the spectral region of interest for this exemplary case. As shown in FIG. 1, the TM and SC are located in the anterior chamber of the eye between the iris and directly beneath the corneoscleral junction (or limbus) that is nominally about 500 μm thick and has an optical scattering cross-section, μs, of about 40 cm⁻¹ at about 550 nm. To a first order, this yields an irradiance at the target surface (e.g. TM) that is about 9% that at the ocular surface. Thus, for this exemplary case, an incident exposure range of about 11 mW/mm² to about 110 mW/mm² is required for a therapeutic exposure of the TM between about 1 mW/mm² to about 10 mW/mm². These irradiance levels may be achieved to utilize solar radiation to activate NpHR, as described above, by utilizing an optical system with an inverse magnification factor ranging from about 11× to about 110×. This may be accomplished by, for example, using lenses whose diameters range from about 3.6 mm to about 11 mm to produce a 1 mm2 spot at a therapeutic exposure level within the spectral region of interest. A plurality of such lenses, and/or other optical elements capable of providing the requisite inverse magnification factors, such as, but not limited to, those described elsewhere herein may be used to irradiate larger portions of the target tissue. Other exemplary configurations and embodiments are described elsewhere herein.

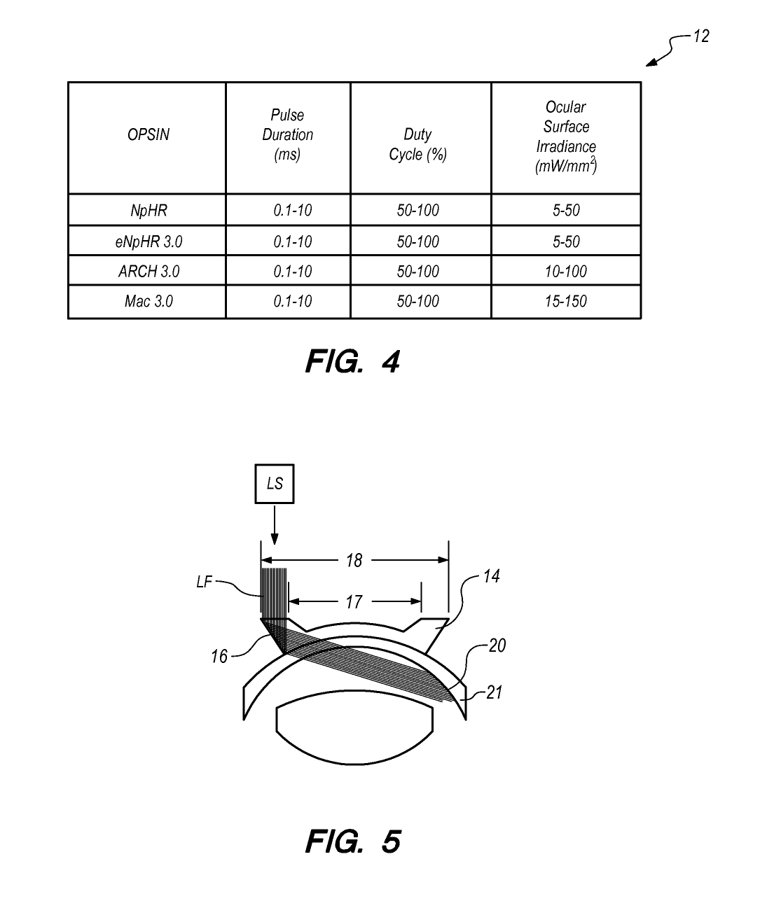

The optical parameters required to effectively activate specific optogenetic targets are listed in the table of FIG. 4. Tissue clearing, or optical clearing as it is also known, may be employed, as well. Tissue clearing refers to the reversible reduction of the optical scattering by a tissue due to refractive index matching of scatterers and ground matter. This may be accomplished by impregnating tissue with substances (“clearing agents”) such as, x-ray contrast agents (e.g. Verografin, Trazograph, and Hypaque-60), glucose, propylene glycol, polypropylene glycol-based polymers (PPG), polyethylene glycol (PEG), PEG-based polymers, and glycerol by way of non-limiting examples. It may also be accomplished by mechanically compressing the tissue. For example, topical application of PEG-400 and Thiazone in a ratio of 9:1 for between 15-60 minutes may be used to decrease the scattering of the sclera to improve the overall transmission of light to the TM.

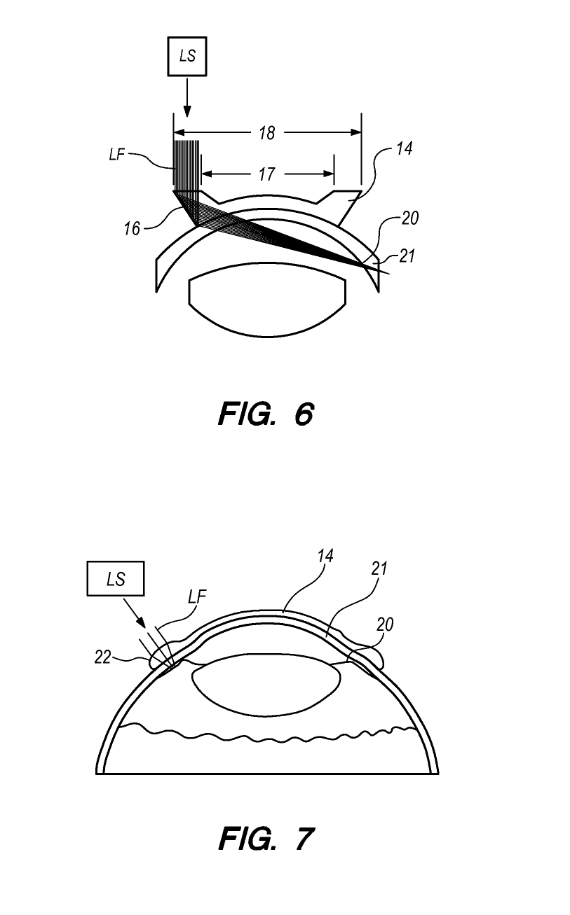

Alternately, the TM may be illuminated using an external light source and/or with concentration of light. The illumination can either be applied directly at some oblique angle to avoid adjacent and/or intermediate tissue(s), or indirectly through the adjacent and/or intermediate tissue(s). Examples of such adjacent and/or intermediate tissue(s) are; the cornea and its constituents, the sclera and its constituents, the limbus and its constituents, the iris and its constituents, the ciliary apparatus, and the lens apparatus. An embodiment is directed at a specialized contact lens with reflective feature(s) on an outer portion to direct light through the cornea, and across the pupil into the target tissue. The central area of the contact lens may allow for normal vision by providing no optical power, or may be configured as a prescription lens, such as is shown in FIG. 5, which features certain optical details. The reflective feature may have a radius of curvature selected such that the light is focused and more concentrated at the target tissue location, such as is shown in FIG. 6, which also depicts certain optical details.

Light also may be applied indirectly through the sclera/limbus. The sclera/limbus is not transparent and a degree of irradiance attenuation will be experienced, as was described above. Light at the source may need to have a high irradiance, or the light may need to be focused or concentrated to achieve the desired irradiance at the target. Alternatively, the sclera could be optically cleared.

A scleral lens may be a large lens that rests on the sclera and may create a tear-filled vault over the cornea. Currently, scleral lenses may be used to improve vision and reduce pain and light sensitivity for people suffering from growing number of disorders or injuries to the eye, such as microphthalmia, keratoconus, corneal ectasia, Stevens-Johnson syndrome, Sjögren's syndrome, aniridia, neurotrophic keratitis, post-LASIK complications, post-corneal transplant complications, and pellucid degeneration. Simple scleral shells to treat these conditions are currently sold through the Boston Foundation for Sight under the trade name PROSE. Unlike the present invention, no currently available scleral lenses make use of peripheral optical elements to direct light into the eye, let alone the sclera, or trabecular meshwork.

One embodiment of the present invention is a contact lens configured to cover the sclera/limbus region. Light may be directed through this region. The contact lens may have a feature such that the light may be focused or concentrated at the target. This feature could be refractive in nature, such as a microlens. The lens may focus an externally applied light though the sclera. The light focusing or concentration feature on the contact lens may also be a diffractive optical element, or, alternately, a Fresnel surface. FIGS. 5-9 illustrate different embodiments of this configuration, wherein a corneosclero contact lens 14 is configured to include a peripheral optical element 16 that may serve to direct light onto the target tissue 20 through the cornea 21, and/or the limbus, and/or the sclera. FIG. 6 shows an embodiment similar to that of FIG. 5, with the addition of optical element 16 also possessing optical power to concentrate light onto the target tissue 20 through the cornea 21, and/or the limbus, and/or the sclera.

Alternately, an optical element or segment, having optical power may be placed at or nearby the outer periphery of contact lens 14 to direct light through the cornea 21, and/or the limbus, and/or the sclera. Such a peripheral lens may be a substantially annular array of lenses, or an annular lens, or a toroidal lens, by way of non-limiting example, with a primary focal length of between 1-3 mm, such as is illustrated as Focusing Feature 22 in FIG. 7.

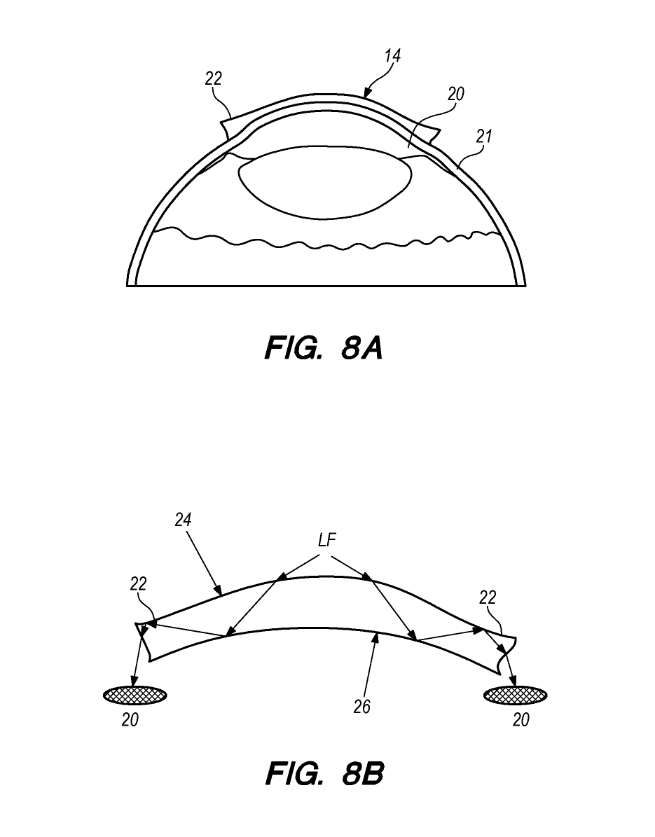

Regarding FIG. 8, anterior surface 24 and posterior surface 26 of contact lens 14 may be designed in a way that light source LS to provide a relatively large spot size and/or of a specific spectrum may be transmitted through the anterior surface 24 of the contact lens 14, and reflected and/or guided toward the periphery, where it may exit the contact lens 14 and illuminate onto Target Tissue 20, nearby the corneoscleral limbus, including, but not limited to, the sclera and cornea. Such a light source may provide for ease of use by means of relatively loose alignment tolerances. The light source may be a narrow band source matched to activate the opsin. The anterior surface of the contact lens may be designed so photons from the external light source refract and reach the posterior surface of the lens within a controlled range of angle of incidence (AOI). The radius of curvature of the posterior surface of the lens may be designed to satisfy the condition for total internal refraction (TIR) for light to be guided toward the end, or it may be coated with reflective coating so light bounces off and exits onto the side. The reflective coating may be reflective for only a certain spectral region, such as one that matches the target activation spectrum. The target illumination pattern thus generated resembles a ring of light that may superimpose onto the target tissue 20 after traversing the outer layers of the eye.

Such a lens system may be further configured to comprise a light sources (or a plurality of light sources) within it. An exemplary embodiment is directed at the use of LEDs embedded within the scleral lens, and configured to obtain power wirelessly from a power transmission source. The power transmission source may be such as those described in the aforementioned incorporated by reference PCT patent application, and may be configured to reside within a hat or shirt, or other garment, that the patient wears. A controller may also be utilized to control the light source(s) by means of modulating their output power, and/or intensity, and/or irradiance, and/or fluence, and/or pulse duration, and/or pulse frequency, and/or duty cycle.

A target illumination contact lens may also be configured to match the prescription of a lens that a may patient wear otherwise for vision correction. Alternately, a 0-power (null) contact lens may be used in order to substantially not alter a patient's vision. In this way the patient doesn't always have to put on and remove the lens just for the purpose of treatment.

In order to not interfere with natural vision illumination, the design of the contact lens may only allow the light guiding function for a specific spectral range and/or a specific range of Angle of Incidience (AOI). Therefore, as shown in FIGS. 8A and 8B, the light source may be coupled with contact lens 14 and only a very small portion of the natural light spectrum is directed to the target tissue 20, e.g. the Trabecular Meshwork.

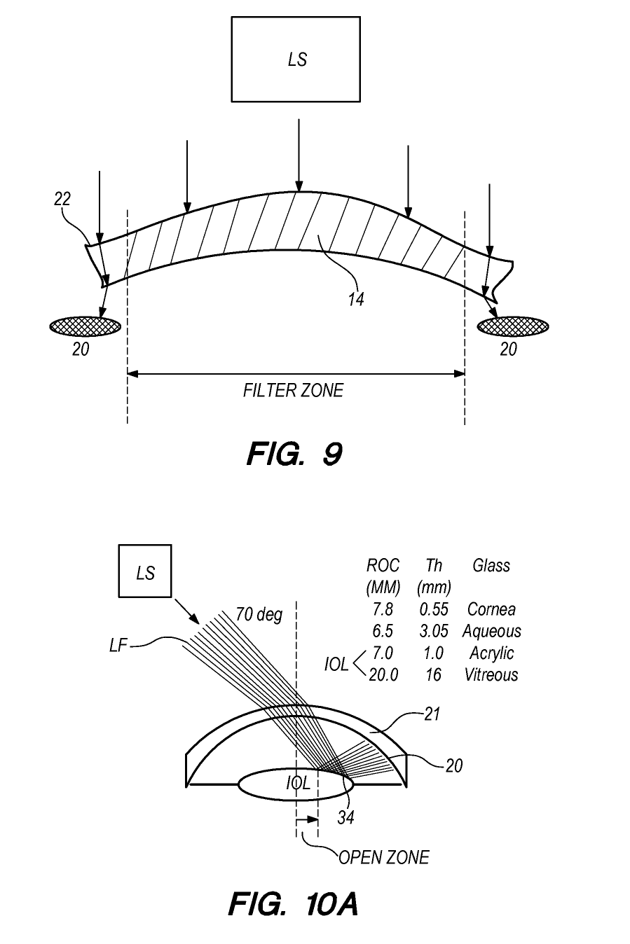

Alternatively, a contact lens may be configured to utilize a filter block integrated in the central portion of the lens, and only the peripheral aspect of the lens may allow transmission and concentration of light from the therapeutic light source, thus forming a light ring onto the TM by traversing through the outer layer(s) of the eye. The light usage may be decreased but it ensures a majority amount of the therapeutic light may be blocked and substantially not allowed to reach the retina by virtue of the spectrally-specific blocking filter which is disposed within contact lens 14, and/or atop the cornea and pupil (labeled as FILTER ZONE), such as is shown in FIG. 9. The filter may be configured to nominally block the therapeutic spectrum from Light Source LS. It may alternately be a photopically neutral filter that removes selected portions of the illumination spectrum, including the therapeutic spectrum, in order to achieve more white-balance.

By way of non-limiting example, materials for the construction of scleral lenses may be selected from the group consisting of; syloxanylstyrene, fluoromethacrylate, roflufocon, silicone hydrogel, fluorosilicone and hydrophilic monomers, fluorosilicone acrylate, fluorinated ethylenepropylene, polymethylpentene, polydimethylsiloxane, polymethylmethacrylate, polyethylene, polypropylene, and THV fluoropolymer blend. These materials are biocompatible and many are used in FDA-cleared extended wear ophthalmic products. They are available from manufacturers and suppliers such as, Contamac, ART Optical, Polymer Technology, The LifeStyle Company, GT Labs, Paragon Vision Sciences, InnoVision, Stellar, and Lagado/Menicon.

Gas permeability (GP) is a feature of certain embodiments of the present invention. GP lenses allow oxygen to pass through the lens and reach the cornea. For example, TYRO-97 from Lagado/Menicon combines high oxygen permeability (Dk=0.97 cm²*mlO²/s*ml*mmHg) for eye health, moderate structural rigidity (Modulus=0.3 MPa) for comfort, with a refractive index of 1.45 at 550 nm. An entire lens may be made from a GP material, or, alternately, a portion of the lens may be made from a GP material. An embodiment is directed at a lens configured to utilize a GP material for the corneal portion/section/segment of the lens and a non-GP material for a more peripheral portion/section/segment of the lens. The non-GP materials may have a higher refractive index than the GP material(s), which may improve the optical power and/or minimize the surface sagittal and/or radii of the therapeutic light collection features of the lens.

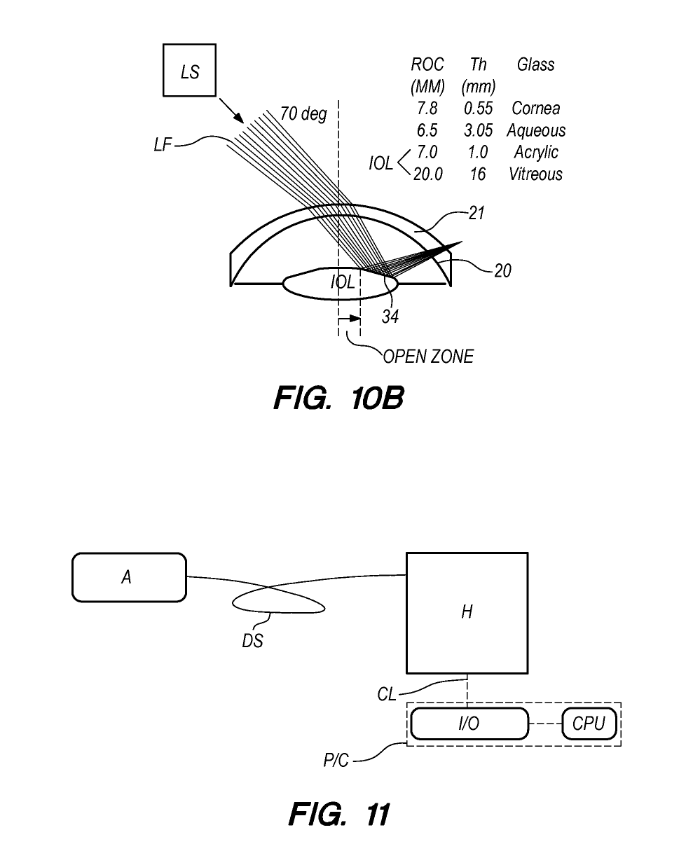

FIGS. 10A and 10B illustrate alternate embodiments directed at systems configured to utilize an implanted Intraocular Lens (IOL). The IOL may contain a feature that enhances the amount of light that may be directed into the target tissue 20, such as Reflective Surface 34. This feature on the IOL may be a coating that covers substantially all of the optical surface(s) of the IOL and directs a portion of the incident light spectrum onto the target tissue 20 while passing the balance of the spectrum towards the retina such that the perception of the light is substantially white balanced. Alternatively, the feature on the IOL could be wavelength selective as a function of position. That is, only the edges of the IOL could be reflective for the therapeutic light only, while the remaining optical surface(s) of the IOL are otherwise uncoated. This configuration may used to utilize ambient light. Alternately, it may utilize an external light source that may be configured relative to the IOL such that its light is reflected by the IOL and into the direction of the TM. FIGS. 10A and 10B illustrate the use of focused and unfocused light with these embodiments, and include optical prescriptions for their construction.

Mitigation configurations may be included to prevent damage to tissue not intentionally targeted. Namely, there could be damage to the retina or iris or other structures in the eye if the light is directed onto them for long periods of time and/or at illumination levels over 1 mW in the visible spectrum. To accommodate this, the system may be configured to be responsive in the sense that it will shut off light output when a misaligned condition is detected. The misalignment could be detected by using eye-tracking techniques configured to recognize the iris, for example. Such eye-tracking systems are utilized in the products sold by Sensomotoric Instruments, and described by Hohla et al, in U.S. Pat. No. 7,146,983, which is incorporated by reference herein in its entirety. A therapeutic light source may be configured to work with an eye tracking system to deliver light only when the patient is looking in a direction that is aligned for delivery of the therapeutic light. Alternately, the therapeutic light source may be scanned to provide more access to the TM, and configured to receive positional and/or directional input from the eye tracking system.

Referring to FIG. 11, a suitable light delivery system comprises one or more applicators (A) configured to provide light output to the targeted tissue structure(s), such as the Trabecular Meshwork and/or Schlemm's Canal. The light may be generated within the applicator (A) structure itself, or within a housing (H) that is operatively coupled to the applicator (A) via one or more delivery segments (DS), or at a location between the housing (H) and the applicator (A). The one or more delivery segments (DS) serve to transport, or guide, the light to the applicator (A) when the light is not generated in the applicator itself. In an embodiment wherein the light is generated within the applicator (A), the delivery segment (DS) may simply comprise an electrical connector to provide power to the light source and/or other components which may be located distal to, or remote from, the housing (H). The one or more housings (H) preferably are configured to serve power to the light source and operate other electronic circuitry, including, for example, telemetry, communication, control and charging subsystems. External programmer and/or controller (P/C) devices may be configured to be operatively coupled to the housing (H) from outside of the patient via a communications link (CL), which may be configured to facilitate wireless communication or telemetry, such as via transcutaneous inductive coil configurations, between the programmer and/or controller (P/C) devices and the housing (H). The programmer and/or controller (P/C) devices may comprise input/output (I/O) hardware and software, memory, programming interfaces, and the like, and may be at least partially operated by a microcontroller or processor (CPU), which may be housed within a personal computing system which may be a standalone system, or be configured to be operatively coupled to other computing or storage systems. Such systems are described in U.S. Pat. Appl. No. [CKT 20034.40], which shares at least one inventor in common with the present application, and is included by reference in its entirety herein.

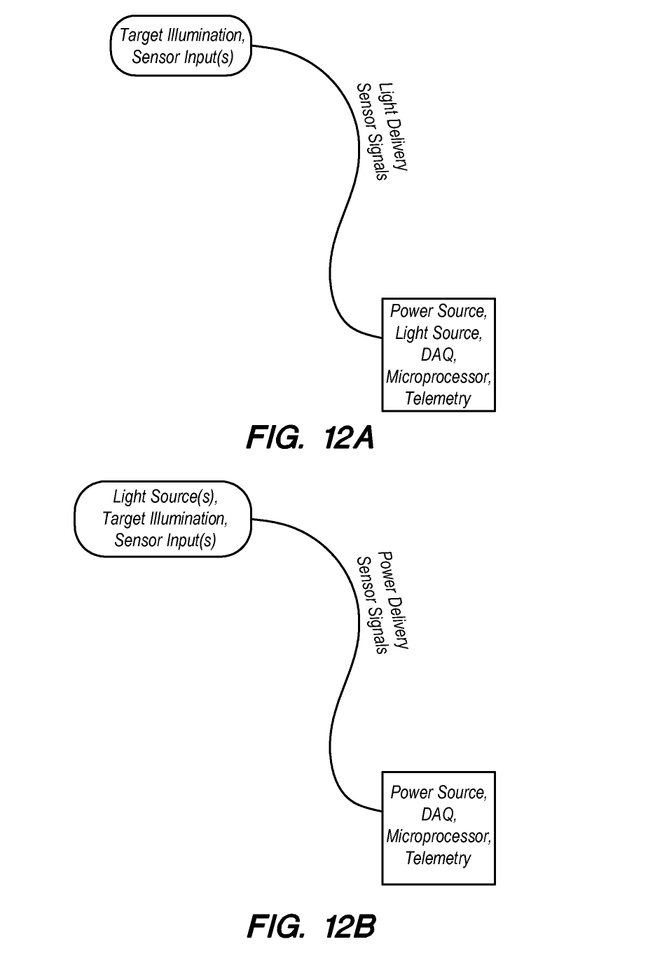

A variety of embodiments of light applicators are and system architectures are disclosed herein, and in the aforementioned included reference. There are further bifurcations that depend upon where the light is produced (i.e., in or near the applicator vs. in the housing or elsewhere). FIGS. 12A and 12B illustrate these two configurations.

Referring to FIG. 12A, in a first configuration, light is generated in the housing and transported to the applicator via the delivery segment. The delivery segment(s) may be optical waveguides, selected from the group consisting of round fibers, hollow waveguides, holey fibers, photonic bandgap devices, and/or slab configurations, as have described previously. Multiple waveguides may also be employed for different purposes. As a non-limiting example, a traditional circular cross-section optical fiber may be used to transport light from the source to the applicator because such fibers are ubiquitous and may be made to be robust and flexible. Alternately, such a fiber may be used as input to another waveguide, this with a polygonal cross-section providing for regular tiling. Such waveguides have cross-sectional shapes that pack together fully, i.e. they form an edge-to-edge tiling, or tessellation, by means of regular congruent polygons. That is, they have the property that their cross-sectional geometry allows them to completely fill (pack) a two-dimensional space. This geometry yields the optical property that the illumination may be made to spatially homogeneous across the face of such a waveguide. Complete homogeneity is not possible with other geometries, although they may be made to have fairly homogeneous irradiation profiles nonetheless. For the present application, a homogenous irradiation distribution is useful because it may provide for uniform illumination of the target tissue. Thus, such regular-tiling cross-section waveguides may be useful. It is also to be understood that this is a schematic representation and that multiple applicators and their respective delivery segments may be employed. Alternately, a single delivery segment may service multiple applicators, or comprise multiple emission locations, as is described elsewhere herein and in the aforementioned included reference. Similarly, a plurality of applicator types may also be employed.

Referring to the configuration of FIG. 12B, light is in the applicator. The power to generate the optical output is contained within the housing and is transported to the applicator via the delivery segment. It is to be understood that this is a schematic representation and that multiple applicators and their respective delivery segments may be employed. Similarly, a plurality of applicator types may also be employed.

The pertinent delivery segments may be optical waveguides, such as optical fibers, in the case where the light is not generated in or near the applicator(s). Alternately, when the light is generated at or near the applicator(s), the delivery segments may be electrical wires. They may be further comprised of fluidic conduits to provide for fluidic control and/or adjustment of the applicator(s). They may also be any combination thereof, as dictated by the specific embodiment utilized, as have been previously described.

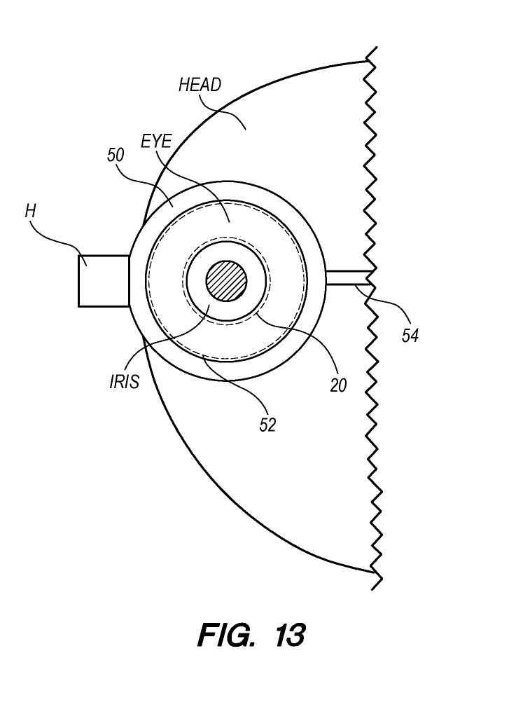

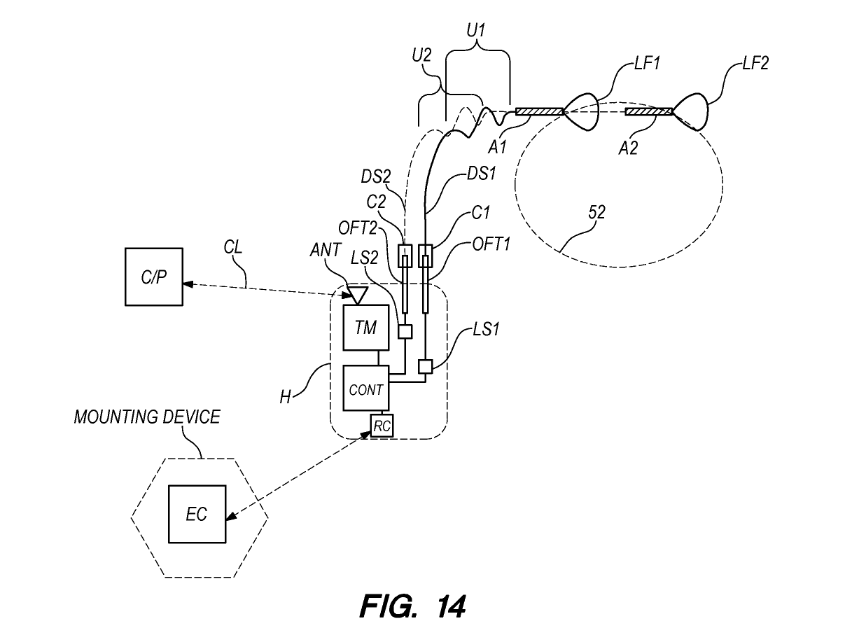

The schematic representation of FIG. 13 depicts a configuration comprising components and sub-systems consistent with that described with respect to FIGS. 2, and 5-12. In this embodiment, the therapeutic device consisting of an array Applicator(s) A 52 that are disposed about ring (and connected to Ring 50 via Delivery Segment(s) DS (not shown) and Housing H is contained within a structural member nominally adjacent to Ring 50 that surrounds the EYE of a patient. The device may be worn on the head of the patient, like eyeglasses, and include Bridge 54 for that purpose. Alternately, Housing H may be located elsewhere in or substantially near the body of the patient, and connected to Applicator(s) A within the brain of the patient via Delivery Segment(s) DS. Ring 50 may be configured to communicate with a controller within housing H, as has been described in detailed elsewhere herein.

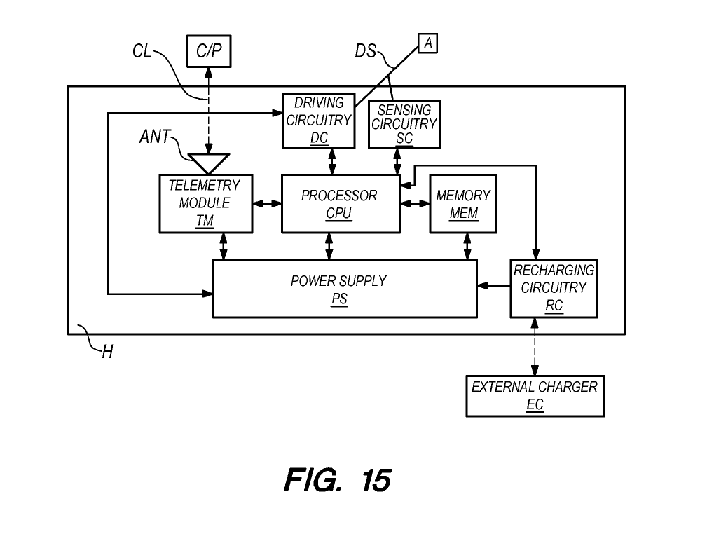

FIG. 14 shows an exemplary embodiment of a system for the treatment of OHT via optogenetic control, configured for therapeutic use as described with respect to FIGS. 11, 12A, & 13. Applicators A1 & A2, may be end-emitting-type applicators that are nominally comprised of optical waveguides, such as optical fiber(s), and are deployed within the Ring 50 to form Array 52, such as is described in more detail with respect to FIG. 13. Light is delivered to Applicators A1 & A2 via Delivery Segments DS1 & DS2, respectively, to create Light Fields LF1 & LF2, respectively, within the target tissues. Light Fields LF1 & LF2 may be configured to provide illumination of the target tissues within the intensity range of 0.01-10 mW/mm², and may be dependent upon one or more of the following factors; the specific opsin used, it's concentration distribution within the tissue, the tissue optical properties, and the size of the target structure(s). Although not shown for simplicity and clarity in the present figure, multiple applicators and/or delivery segments may be used for a specific target structure if it is a large target structure when compared to the optical extent within that target structure, or if multiple target structures are considered. Likewise, a single light source and applicator are also considered herein and within the scope of the present invention. Delivery Segments DS1 & DS2 may be configured to be optical fibers, such as 105 μm core diameter/125 μm cladding diameter/225 μm acrylate coated 0.22 NA step index fiber that is enclosed in a protective sheath, such as a 300 μm OD silicone tube. Connectors Cl & C2 are configured to operatively couple light from Delivery Segments DS1 & DS2 to Applicators A1 & A2, respectively. Delivery Segments DS1 & DS2 further comprise Undulations U1 & U2, respectively, which may provide strain relief. Delivery Segments DS1 & DS2 are operatively coupled to Housing H via Optical Feedthroughs OFT1 & OFT2, respectively. Light is provided to Delivery Segments DS1 & DS2 from Light Sources LS1 & LS2, respectively, within Housing H. Light Sources LS1 & LS2 may be configured to be LEDs, and/or lasers that provide spectrally different output to activate and/or deactivate the opsins resident within target tissue(s), as dictated by the therapeutic paradigm. For example, LS1 may be configured to be a blue laser source, such as the LD-445-20 from Roithner Lasertechnik that produces up to 20 mW of 450 nm light, and is suitable for use in optogenetic intervention using such opsins as ChR2, and/or iChR2. Light Source LS2 may be configured to be a different laser than LS1, such as the QLD0593-9420 from QD Photonics that produces up to 20 mW of 589 nm light, and is suitable for use in optogenetic inhibition using NpHR. Light Sources LS1 & LS2 may be independently controlled by controller CONT, such that the exposures provided by Light Fields LF1 & LF2 are configured independently for response of their respective target tissue. The Controller CONT shown within Housing H is a simplification, for clarity, of that described in more detail with respect to FIG. 15. External clinician programmer module and/or a patient programmer module C/P may communicate with Controller CONT via Telemetry module TM via Antenna ANT via Communications Link CL. Power Supply PS, not shown for clarity, may be wirelessly recharged using External Charger EC. Furthermore, External Charger EC may be configured to reside within a Mounting Device MOUNTING DEVICE. Mounting Device MOUNTING DEVICE may be a hat, as is especially well configured for this exemplary embodiment. External Charger EC, as well as External clinician programmer module and/or a patient programmer module C/P and Mounting Device MOUNTING DEVICE may be located within the extracorporeal space.

Referring to FIG. 15, a schematic is depicted illustrating various components of an example housing H. In this example, stimulator includes processor CPU, memory M, power source PS, telemetry module TM, antenna ANT, and the driving circuitry DC for an optical stimulation generator (which may or may not include a light source, as has been previously described). The Housing H is shown coupled to one Delivery Segments DSx for simplicity and clarity. It may be a multi-channel device in the sense that it may be configured to include multiple optical paths (e.g., multiple light sources and/or optical waveguides or conduits) that may deliver different optical outputs, some of which may have different wavelengths. More or less delivery segments may be used in different implementations, such as, but not limited to, one, two, five or more optical fibers and associated light sources may be provided. The delivery segments may be detachable from the housing, or be fixed.

Memory (MEM) may store instructions for execution by Processor CPU, optical and/or sensor data processed by sensing circuitry SC, and obtained from sensors both within the housing, such as battery level, discharge rate, etc., and those deployed outside of the Housing (H), possibly in Applicator A, such as optical and temperature sensors, and/or other information regarding therapy for the patient. Processor (CPU) may control Driving Circuitry DC to deliver power to the light source (not shown) according to a selected one or more of a plurality of programs or program groups stored in Memory (MEM). The Light Source may be internal to the housing H, or remotely located in or near the applicator (A), as previously described. Memory (MEM) may include any electronic data storage media, such as random access memory (RAM), read-only memory (ROM), electronically-erasable programmable ROM (EEPROM), flash memory, etc. Memory (MEM) may store program instructions that, when executed by Processor (CPU), cause Processor (CPU) to perform various functions ascribed to Processor (CPU) and its subsystems, such as dictate pulsing parameters for the light source.

In accordance with the techniques described in this disclosure, information stored in Memory (MEM) may include information regarding therapy that the patient had previously received. Storing such information may be useful for subsequent treatments such that, for example, a clinician may retrieve the stored information to determine the therapy applied to the patient during his/her last visit, in accordance with this disclosure. Processor CPU may include one or more microprocessors, digital signal processors (DSPs), application-specific integrated circuits (ASICs), field-programmable gate arrays (FPGAs), or other digital logic circuitry. Processor CPU controls operation of stimulator, e.g., controls stimulation generator to deliver stimulation therapy according to a selected program or group of programs retrieved from memory (MEM). For example, processor (CPU) may control Driving Circuitry DC to deliver optical signals, e.g., as stimulation pulses, with intensities, wavelengths, pulse widths (if applicable), and rates specified by one or more stimulation programs. Processor (CPU) may also control Driving Circuitry (DC) to selectively deliver the stimulation via subsets of Delivery Segments (DSx), and with stimulation specified by one or more programs. Different delivery segments (DSx) may be directed to different target tissue sites, as was previously described.

Telemetry module (TM) may include a radio frequency (RF) transceiver to permit bi-directional communication between stimulator and each of clinician programmer and patient programmer (C/P). Telemetry module (TM) may include an Antenna (ANT), of any of a variety of forms. For example, Antenna (ANT) may be formed by a conductive coil or wire embedded in a housing associated with medical device. Alternatively, antenna (ANT) may be mounted on a circuit board carrying other components of stimulator or take the form of a circuit trace on the circuit board. In this way, telemetry module (TM) may permit communication with a controller/programmer (C/P). Given the energy demands and modest data-rate requirements, the Telemetry system may be configured to use inductive coupling to provide both telemetry communications and power for recharging, although a separate recharging circuit (RC) is shown in FIG. 15 for explanatory purposes.

External programming devices for patient and/or physician can be used to alter the settings and performance of the implanted housing. Similarly, the implanted apparatus may communicate with the external device to transfer information regarding system status and feedback information. This may be configured to be a PC-based system, or a stand-alone system. In either case, the system must communicate with the housing via the telemetry circuits of Telemetry Module (TM) and Antenna (ANT). Both patient and physician may utilize controller/programmers (C/P) to tailor stimulation parameters such as duration of treatment, optical intensity or amplitude, pulse width, pulse frequency, burst length, and burst rate, as is appropriate.

Once the communications link (CL) is established, data transfer between the MMN programmer/controller and the housing may begin. Examples of such data are:
1. From housing to controller/programmer:
   a. Patient usage
   b. Battery lifetime
   c. Feedback data
      i. Device diagnostics (such as direct optical transmission measurements by an emitter-opposing photosensor)
2. From controller/programmer to housing:
   a. Updated illumination level settings based upon device diagnostics
   b. Alterations to pulsing scheme
   c. Reconfiguration of embedded circuitry
      i. FPGA, etc.

By way of non-limiting examples, near field communications, either low power and/or low frequency; such as is produced by Zarlink/MicroSEMI.

Various exemplary embodiments of the invention are described herein. Reference is made to these examples in a non-limiting sense. They are provided to illustrate more broadly applicable aspects of the invention. Various changes may be made to the invention described and equivalents may be substituted without departing from the true spirit and scope of the invention. In addition, many modifications may be made to adapt a particular situation, material, composition of matter, process, process act(s) or step(s) to the objective(s), spirit or scope of the present invention. Further, as will be appreciated by those with skill in the art that each of the individual variations described and illustrated herein has discrete components and features which may be readily separated from or combined with the features of any of the other several embodiments without departing from the scope or spirit of the present inventions. All such modifications are intended to be within the scope of claims associated with this disclosure.

Any of the devices described for carrying out the subject diagnostic or interventional procedures may be provided in packaged combination for use in executing such interventions. These supply “kits” may further include instructions for use and be packaged in sterile trays or containers as commonly employed for such purposes.

The invention includes methods that may be performed using the subject devices. The methods may comprise the act of providing such a suitable device. Such provision may be performed by the end user. In other words, the “providing” act merely requires the end user obtain, access, approach, position, set-up, activate, power-up or otherwise act to provide the requisite device in the subject method. Methods recited herein may be carried out in any order of the recited events which is logically possible, as well as in the recited order of events.

Exemplary aspects of the invention, together with details regarding material selection and manufacture have been set forth above. As for other details of the present invention, these may be appreciated in connection with the above-referenced patents and publications as well as generally known or appreciated by those with skill in the art. The same may hold true with respect to method-based aspects of the invention in terms of additional acts as commonly or logically employed.

In addition, though the invention has been described in reference to several examples optionally incorporating various features, the invention is not to be limited to that which is described or indicated as contemplated with respect to each variation of the invention. Various changes may be made to the invention described and equivalents (whether recited herein or not included for the sake of some brevity) may be substituted without departing from the true spirit and scope of the invention. In addition, where a range of values is provided, it is understood that every intervening value, between the upper and lower limit of that range and any other stated or intervening value in that stated range, is encompassed within the invention.

Also, it is contemplated that any optional feature of the inventive variations described may be set forth and claimed independently, or in combination with any one or more of the features described herein. Reference to a singular item, includes the possibility that there are plural of the same items present. More specifically, as used herein and in claims associated hereto, the singular forms “a,” “an,” “said,” and “the” include plural referents unless the specifically stated otherwise. In other words, use of the articles allow for “at least one” of the subject item in the description above as well as claims associated with this disclosure. It is further noted that such claims may be drafted to exclude any optional element. As such, this statement is intended to serve as antecedent basis for use of such exclusive terminology as “solely,” “only” and the like in connection with the recitation of claim elements, or use of a “negative” limitation.

Without the use of such exclusive terminology, the term “comprising” in claims associated with this disclosure shall allow for the inclusion of any additional element—irrespective of whether a given number of elements are enumerated in such claims, or the addition of a feature could be regarded as transforming the nature of an element set forth in such claims. Except as specifically defined herein, all technical and scientific terms used herein are to be given as broad a commonly understood meaning as possible while maintaining claim validity.

The breadth of the present invention is not to be limited to the examples provided and/or the subject specification, but rather only by the scope of claim language associated with this disclosure.

What is claimed:

1. A system for treating hypertension within the eye of a patient, comprising:
a light delivery element optically intercoupled between a light source and a targeted tissue structure of the eye;
wherein the targeted tissue structure has been genetically modified to have light sensitive protein; and
wherein exposure of the genetically modified targeted tissue structure to radiation delivered through the light delivery element modulates the contractility of the genetically modified targeted tissue structure to reduce an intraocular pressure within the eye.

2. The system of claim 1, wherein the light delivery element comprises a contact lens configured to directly interface with a corneal surface of the eye.

3. The system of claim 2, wherein the contact lens is configured to direct light incoming toward the eye to a structure selected from the group consisting of: a trabecular meshwork of the eye; and a schlemm's canal of the eye.

4. The system of 3, wherein the contact lens is configured to direct light incoming toward the eye through an intermediate tissue structure selected from the group consisting of: a cornea of the eye; a limbus of the eye; and a sclera of the eye.

5. The system of claim 2, wherein the contact lens is further configured to comprise an optical element or segment configured to reflect incoming light.

6. The system of claim 2, wherein the contact lens is further configured to comprise an optical element or segment configured to focus incoming light.

7. The system of claim 2, wherein the contact lens is further configured to comprise an optical element or segment configured to both reflect and focus incoming light.

8. The system of claim 2, wherein the contact lens is further configured to comprise a spectral filtering element.

9. The system of claim 1, wherein the light delivery element comprises an intraocular lens configured to redirect at least a portion of radiation incoming toward the eye to the targeted tissue structure of the eye.

10. The system of claim 9, wherein the intraocular lens comprises an implantable prosthesis.

11. The system of claim 9, wherein the intraocular lens is configured to direct light incoming toward the eye to a structure selected from the group consisting of: a trabecular meshwork of the eye; and a schlemm's canal of the eye.

12. The system of 11, wherein the intraocular lens is configured to direct light incoming toward the eye through an intermediate tissue structure selected from the group consisting of: a cornea of the eye; a limbus of the eye; and a sclera of the eye.

13. The system of claim 9, wherein the intraocular lens is further configured to comprise an optical element or segment configured to reflect incoming light.

14. The system of claim 9, wherein the intraocular lens is further configured to comprise an optical element or segment configured to focus incoming light.

15. The system of claim 9, wherein the intraocular lens is further configured to comprise an optical element or segment configured to both reflect and focus incoming light.

16. The system of claim 1, wherein the light source is coupled to a mounting structure configured to be removably coupled to the patient.

17. The system of claim 16, wherein the mounting structure comprises an eyeglasses frame.

18. The system of claim 1, wherein the light source comprises ambient light from an environment around the patient.

19. The system of claim 1, wherein the light sensitive protein is an opsin protein.

20. The system of claim 19, wherein the opsin protein is an inhibitory opsin protein.

21. The system of claim 20, wherein the inhibitory opsin protein is selected from the group consisting of: NpHR, eNpHR 1.0, eNpHR 2.0, eNpHR 3.0, Mac, Mac 3.0, Arch, ArchT, and iChR.

22. The system of claim 19, wherein the opsin protein is a stimulatory opsin protein.

23. They system of claim 22, wherein the stimulatory opsin protein is selected from the group consisting of: ChR2, C1V1-T, C1V1-TT, CatCh, VChR1-SFO, and ChR2-SFO.

24. The system of claim 1, wherein the light source is configured to produce pulses of light to be delivered to the targeted tissue structure of the eye.
